(19) United States
(12) Patent Application Publication
(10) Pub. No.: US 2024/0301179 A1
(43) Pub. Date: Sep. 12, 2024

(54) POLYAMINO IODINATED COMPOUNDS AND RADIOPAQUE HYDROGELS FORMED THEREFROM

(71) Applicant: Boston Scientific Scimed, Inc., Maple Grove, MN (US)

(72) Inventors: Yen-Hao Hsu, Shrewsbury, MA (US); Cristian Parisi, Boston, MA (US); Joseph Thomas Delaney, JR., Minneapolis, MN (US)

(73) Assignee: Boston Scientific Scimed, Inc., Maple Grove, MN (US)

(21) Appl. No.: 18/591,870

(22) Filed: Feb. 29, 2024

Related U.S. Application Data
(60) Provisional application No. 63/488,080, filed on Mar. 2, 2023.

(51) Int. Cl. C08L 5/08; A61K 9/06; A61K 47/36; A61L 27/18; A61L 27/20; A61L 27/50; A61L 27/52; A61L 27/58; C08J 3/075

(52) U.S. Cl. CPC C08L 5/08; A61K 9/06; A61K 47/36; A61L 27/18; A61L 27/20; A61L 27/50; A61L 27/52; A61L 27/58; C08J 3/075; C08J 2305/08; C08L 2201/06; C08L 2203/02; C08L 2312/00

(57) ABSTRACT

In some aspects, the present disclosure pertains to radiopaque crosslinked hydrogel compositions that comprise a crosslinked reaction product of (a) a polyamino iodinated compound and (b) a reactive polymer that comprises a plurality of reactive groups that are reactive with the amino groups of the polyamino iodinated compound. In other aspects, the present disclosure pertains to systems for forming a hydrogel composition that comprise (a) a polyamino iodinated compound and (b) a reactive polymer that comprises a plurality of reactive groups that are reactive with the amino groups of the polyamino iodinated compound. In further aspects, the present disclosure pertains to methods of treatment using such systems.

### CROSS-REFERENCE TO RELATED APPLICATIONS

This application claims the benefit of U.S. Provisional Patent Application Ser. No. 63/488,080 filed on Mar. 2, 2023, the disclosure of which is incorporated herein by reference.

### FIELD

The present disclosure relates to iodinated compounds, to hydrogels formed from iodinated compounds, and to methods of making and using iodinated compounds and hydrogels, among other aspects. The iodinated compounds of the present disclosure are useful, for example, in forming hydrogels for various biomedical applications.

### BACKGROUND

Bioresorbable hydrogels with rapid crosslinking reaction rate in vivo, known by the trade name of SpaceOAR®, have become a prominent biomaterial and obtained clinical success in creating the space between prostate and rectum, tremendously improving patient safety during the cancer therapies. A further improvement based on this application is that some of 8-Arm PEG branches are functionalized with 2,3,5-triiiodobenzamide (TIB) groups, replacing part of the activated ester end groups, succinimidyl glutarate (SG), in order to provide intrinsic radiopacity to the hydrogels themselves for CT-visibility. This hydrogel, known by the trade name of SpaceOAR Vue®, is the next generation of SpaceOAR® for prostate medical applications.

While the above approach is effectual, using the arms of the 8-arm polymer to functionalize the hydrogel with iodine means that there are fewer arms available to crosslink. This can be overcome by adding more polymer, but the loading of solids increases, which can adversely impact viscosity. Reducing the molecular weight can cut down on the loading of solids, but this results in a lower melting point, and problems with processability. An additional effect of the reduced crosslink density per 8-arm polymer is that the resulting gel has a slower cure rate, which means the gel is liquid and mobile in vivo for longer time periods, opening up opportunities for unintended side-reactions and material displacement. Moreover, TIB is sparingly water soluble, meaning that there is an upper limit to how much iodine can be added before the solubility of the gel becomes impacted. In the event that the concentration of TIB groups is so high that the star-PEG precipitates out of solution, the TIB groups can physically crosslink the system before it even reacts, requiring greater force to dispense.

There is thus a continuing need in the biomedical arts for additional hydrogels, for precursors of such hydrogels, for methods of making such hydrogels and precursors, for methods of using such hydrogels and precursors, and for systems for forming such hydrogels, among other needs.

The present disclosure provides an alternative approach to that described above. Rather than using the arms of the star polymer to functionalize the hydrogel with iodine, in the present disclosure, the crosslinker for the star polymer is functionalized with iodine.

### SUMMARY

In various aspects, the present disclosure relates to radiopaque crosslinked hydrogel compositions that comprise a crosslinked reaction product of the following: (a) a polyamino iodinated compound and (b) a reactive polymer that comprises a plurality of reactive groups that are reactive with the amino groups of the polyamino iodinated compound.

In some embodiments, which may be used in conjunction with above aspects, the polyamino iodinated compound comprises a polyamino moiety that is linked to an iodinated moiety through an amide group.

In some embodiments, which may be used in conjunction with the above aspects and embodiments, the iodinated moiety may be an iodinated aromatic moiety.

In some embodiments, which may be used in conjunction with the above aspects and embodiments, the iodinated aromatic moiety comprises a monocyclic or multicyclic aromatic structure that is substituted with one or more iodine atoms. In some of these embodiments, the monocyclic or multicyclic aromatic structure is further substituted with one or more hydroxy-containing groups independently selected from hydroxy groups and C₁-C₄-hydroxyalkyl groups.

In some embodiments, which may be used in conjunction with the above aspects and embodiments, the polyamino moiety comprises a plurality of —(CH₂)ₓ—NH₂ groups where x is 0, 1, 2 3, 4, 5 or 6.

In some embodiments, which may be used in conjunction with the above aspects and embodiments, the polyamino iodinated compound comprises a residue of a peptide oligomer that comprises from 2 to 10 lysine and/or ornithine amino acid residues and a residue of an iodinated amine compound.

In some embodiments, which may be used in conjunction with the above aspects and embodiments, the iodinated amine compound comprises a monocyclic or multicyclic aromatic structure substituted with (a) an amine substituent, (b) one or more iodine atoms and (c) optionally, one or more hydroxy-containing groups independently selected from hydroxy groups and C₁-C₄-hydroxyalkyl groups. In some of these embodiments, the amine substituent may be selected from an amino (—NH₂) group and an amino-C₁-C₆-alkyl group.

In some embodiments, which may be used in conjunction with the above aspects and embodiments, the reactive polymer is a reactive multi-arm polymer that comprises a plurality of hydrophilic polymer arms having reactive end groups that are reactive with amino groups of the polyamino iodinated compound.

In some of these embodiments, the hydrophilic polymer arms may comprise one or more hydrophilic monomers selected from ethylene oxide, N-vinyl pyrrolidone, oxazoline monomers, hydroxyethyl acrylate, hydroxyethyl methacrylate, PEG methyl ether acrylate or PEG methyl ether methacrylate, or PNIPAAM and/or the reactive end groups may be linked to the hydrophilic polymer arms by a hydrolysable ester and/or the reactive end groups may be electrophilic groups.

In some embodiments, which may be used in conjunction with the above aspects and embodiments, the reactive polymer comprises carboxylic acid groups that react with the amino groups of the polyamino iodinated compound to form amide linkages.

In some of these embodiments, the reactive polymer is a carboxylic-acid-containing polysaccharide.

In some embodiments, which may be used in conjunction with the above aspects and embodiments, the radiopaque crosslinked hydrogel composition comprises radiopaque particles that comprise the crosslinked reaction product.

In some embodiments, which may be used in conjunction with the above aspects and embodiments, the radiopaque crosslinked hydrogel composition further comprises one or more additional agents selected from therapeutic agents, imaging agents, colorants, tonicity adjusting agents, suspension agents, wetting agents, and pH adjusting agents.

In some aspects, the present disclosure pertains to systems for forming a hydrogel composition that comprises (a) a polyamino iodinated compound in accordance with above aspects and embodiments, and (b) a reactive polymer that comprises a plurality of reactive groups that are reactive with the amino groups of the polyamino iodinated compound in accordance with above aspects and embodiments.

In some embodiments, the system comprises a first composition that comprises the polyamino iodinated compound, a second composition that comprises the reactive polymer, and an optional accelerant composition.

In some embodiments, which may be used in conjunction with the above aspects and embodiments, the system comprises one or more additional agents selected from therapeutic agents, imaging agents, colorants, tonicity adjusting agents, suspension agents, wetting agents, and pH adjusting agents.

In some embodiments, which may be used in conjunction with the above aspects and embodiments, the system further comprises a delivery device.

In some aspects, the present disclosure pertains to methods of treatment comprising administering to a subject a mixture that comprises a polyamino iodinated compound in accordance with above aspects and embodiments and a reactive multi-arm polymer that comprises a plurality of hydrophilic polymer arms having reactive end groups that are reactive with amino groups of the polyamino iodinated compound in accordance with above aspects and embodiments under conditions such that the polyamino iodinated compound and the reactive multi-arm polymer crosslink after administration.

In some aspects, the present disclosure pertains to methods of making a polyamino iodinated compound that comprise (a) forming a protected carboxyl-substituted polyamino compound by protecting amino groups of the carboxyl-substituted polyamino compound, (b) forming an amide linkage between the carboxyl group of the protected carboxyl-substituted polyamino compound and an amino group of an iodinated amine compound, and (c) deprotecting amino groups of the product of step (b).

Potential benefits associated with the present disclosure include one or more of the following: radiocontrast is maintained, complexity and cost of the manufacturing process is reduced, melting point of the solid components of the hydrogel can be maintained above 40° C. (improving storage and handling), homogeneity of the final hydrogel is improved, in vivo persistence is obtained, and cure kinetics are maintained.

The above and other aspects, embodiments, features and benefits of the present disclosure will be readily apparent from the following detailed description.

### BRIEF DESCRIPTION OF THE DRAWINGS

FIG. 1 schematically illustrates a method of forming a polyamino iodinated compound, in accordance with an embodiment of the present disclosure.

FIG. 2 schematically illustrates a method of forming a polyamino iodinated compound, in accordance with another embodiment of the present disclosure.

FIG. 3 schematically illustrates a method of forming a radiopaque crosslinked hydrogel, in accordance with an embodiment of the present disclosure.

FIG. 4 schematically illustrates a method of forming a radiopaque crosslinked polysaccharide hydrogel, in accordance with an embodiment of the present disclosure.

### DETAILED DESCRIPTION

In several aspects of the present disclosure, a radiopaque crosslinked hydrogel is provided that comprises a crosslinked reaction product of (a) a polyamino iodinated compound and (b) a reactive polymer that comprises a plurality of reactive groups that are reactive with the amino groups of the polyamino iodinated compound. Unless indicated otherwise, as used herein the prefix “poly” means two or more.

In various embodiments, the polyamino iodinated compounds of the present disclosure comprise a polyamino moiety that is linked to an iodinated moiety through an amide group. In particular embodiments, detailed below, the polyamino iodinated compounds may comprise peptide oligomers that contain from 2 to 10 lysine and/or ornithine amino acid residues and one or more iodinated amine residues.

Iodinated moieties of the present disclosure include iodinated aromatic moieties. Examples of iodinated aromatic moieties include those that comprise one or more iodinated aromatic groups (also referred to as an iodo-aromatic group). Examples of iodinated aromatic groups include iodine-substituted monocyclic aromatic groups and iodine-substituted multicyclic aromatic groups, such as iodo-phenyl groups, iodo-naphthyl groups, iodo-anthracene groups, iodo-phenanthrene groups, and iodo-tetracene groups.

The aromatic groups may be substituted with one, two, three, four, five, six or more iodine atoms. More particular examples of iodo-aromatic groups include iodo-phenyl groups selected from a mono-iodo-phenyl group, a di-iodo-phenyl group, tri-iodo-phenyl group, a tetra-iodo-phenyl group, a penta-iodo-phenyl group, a mono-iodo-naphthyl group, a di-iodo-naphthyl group, tri-iodo-naphthyl group, a tetra-iodo-naphthyl group, a penta-iodo-naphthyl group, a hexa-iodo-naphthyl group, a hepta-iodo-naphthyl group, a mono-iodo-anthracenyl group, a di-iodo-anthracenyl group, tri-iodo-anthracenyl group, a tetra-iodo-anthracenyl group, a penta-iodo-anthracenyl group, a hexa-iodo-anthracenyl group, a hepta-iodo-anthracenyl group, an octa-iodo-anthracenyl group, a nona-iodo-anthracenyl group, a mono-iodo-phenanthrenyl group, a di-iodo-phenanthrenyl group, tri-iodo-phenanthrenyl group, a tetra-iodo-phenanthrenyl group, a penta-iodo-phenanthrenyl group, a hexa-iodo-phenanthrenyl group, a hepta-iodo-phenanthrenyl group, an octa-iodo-phenanthrenyl group, a nona-iodo-phenanthrenyl group, a mono-iodo-tetracenyl group, a di-iodo-tetracenyl group, tri-iodo-tetracenyl group, a tetra-iodo-tetracenyl group, a penta-iodo-tetracenyl group, a hexa-iodo-tetracenyl group, a hepta-iodo-tetracenyl group, an octa-iodo-tetracenyl group, a nona-iodo-tetracenyl group, a deca-iodo-tetracenyl group and a undeca-iodo-tetracenyl group.

Examples of iodinated aromatic moieties include those that comprise one or more monocyclic or multicyclic aromatic structures, such as a benzene or naphthalene structure, substituted with (a) one or more iodine groups (e.g., one two, three, four, five, six or more iodine atoms) and (b) optionally, one or more hydroxy-containing groups (e.g., one two, three, four, five, six or more hydroxy-containing groups). The hydroxy-containing groups may be independently selected from one or more hydroxy groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from amide groups, amine groups, ether groups, ester groups, or carbonate groups, among others.

Examples of iodinated aromatic moieties include those that comprise hydroxy-iodo-phenyl groups, C₁-C₄-hydroxyalkyl-iodo-phenyl groups, hydroxy-C₁-C₄-hydroxyalkyl-iodo-phenyl groups, hydroxy-iodo-naphthyl groups, C₁-C₄-hydroxyalkyl-iodo-naphthyl groups, and hydroxy-C₁-C₄-hydroxyalkyl-iodo-naphthyl groups.

Particular examples of hydroxyalkyl-iodo-phenyl groups include those selected from a mono-hydroxy-group-containing-mono-iodo-phenyl group, a mono-hydroxy-group-containing-di-iodo-phenyl group, a mono-hydroxy-group-containing-tri-iodo-phenyl group, a mono-hydroxy-group-containing-tetra-iodo-phenyl group, a di-hydroxy-group-containing-mono-iodo-phenyl group, a di-hydroxy-group-containing-di-iodo-phenyl group, a di-hydroxy-group-containing-tri-iodo-phenyl group, a tri-hydroxy-group-containing-mono-iodo-phenyl group, and a tri-hydroxy-group-containing-di-iodo-phenyl group, where “hydroxy-group-containing” independently refers to a hydroxy group or a C₁-C₄-hydroxyalkyl group.

In various embodiments, the iodinated moieties of the present disclosure correspond to residues of iodinated amine compounds.

Examples of iodinated amine compounds include those that comprise one or more monocyclic or multicyclic aromatic structures, such as a benzene or naphthalene structure, substituted with (a) an amine substituent, (b) one or more iodine groups (e.g., one, two, three, four, five, six or more iodine atoms) and (c) optionally, one or more hydroxy-containing groups (e.g., one, two, three, four, five, six or more hydroxy-containing groups). As noted above, hydroxy-containing groups may be independently selected from one or more hydroxy groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), which C₁-C₄-hydroxyalkyl groups may be linked to the monocyclic or multicyclic aromatic structures directly or through any suitable linking moiety, which may be selected, for example, from amide groups, amine groups, ether groups, ester groups, or carbonate groups, among others.

Particular examples of amine-substituted-iodo-aromatic compounds include amine-substituted-mono-iodo-benzene compounds, amine-substituted-di-iodo-benzene compounds, amine-substituted-tri-iodo-benzene compounds, amine-substituted-tetra-iodo-benzene compounds, amine-substituted-penta-iodo-benzene compounds, amine-substituted-mono-iodo-naphthylene compounds, amine-substituted-di-iodo-naphthylene compounds, amine-substituted-tri-iodo-naphthylene compounds, amine-substituted-tetra-iodo-naphthylene compounds, amine-substituted-penta-iodo-naphthylene compounds, amine-substituted-hexa-iodo-naphthylene compounds and amine-substituted-hepta-iodo-naphthylene compounds.

Particular examples of amine-substituted-iodo-aromatic compounds further include amine-substituted-mono-hydroxy-group-containing-mono-iodo-benzene compounds, amine-substituted-mono-hydroxy-group-containing-di-iodo-benzene compounds, amine-substituted-mono-hydroxy-group-containing-tri-iodo-benzene compounds, amine-substituted-mono-hydroxy-group-containing-tetra-iodo-benzene compounds, amine-substituted-di-hydroxy-group-containing-mono-iodo-benzene compounds, amine-substituted-di-hydroxy-group-containing-di-iodo-benzene compounds, amine-substituted-di-hydroxy-group-containing-tri-iodo-benzene compounds, amine-substituted-tri-hydroxy-group-containing-mono-iodo-benzene compounds, and amine-substituted-tri-hydroxy-group-containing-di-iodo-benzene compounds, among others, where “hydroxy-group-containing” independently refers to a hydroxy group or a C₁-C₄-hydroxyalkyl group.

Examples of amine substituents for the preceding iodinated amine compounds include amino (—NH₂) groups and primary amine groups including aminoalkyl groups (e.g., amino-C₁-C₆-alkyl groups), specific examples of which include aminomethyl, aminoethyl, aminopropyl, aminobutyl, aminopentyl and aminohexyl groups.

Specific examples of iodinated amine compounds include 3-(2,3,4,5,6-pentaiodophenyl)propan-1-amine, 4-(2,3,4,5,6-pentaiodophenyl)butan-1-amine, and 5-(2,3,4,5,6-pentaiodophenyl)pentan-1-amine, among others.

As detailed further below, the polyamino iodinated compounds of the present disclosure may be formed by an amide coupling reaction between an iodinated amine compound, which can be selected, for example, from any of the preceding iodinated amine compounds, among others, and a carboxyl-substituted polyamino compound, which can be selected, for example, from those described below (after protection of the amino groups), among others. After coupling, the protective groups on the residue of the carboxyl-substituted polyamino compound are removed, thereby providing the final polyamino iodinated compound.

As previously noted, in addition to an iodinated moiety, such as one of those described above, the polyamino iodinated compounds of the present disclosure also comprise a polyamino moiety that is linked to an iodinated moiety through an amide bond.

The polyamino moiety of the polyamino iodinated compounds of the present disclosure comprises a plurality (two, three, four, five, six, seven, eight, nine, ten or more) amino groups. In various embodiments, the polyamino moiety may comprise a plurality of (two, three, four, five, six, seven, eight, nine, ten or more) —(CH₂)ₓ—NH₂ groups where x is 0, 1, 2 3, 4, 5 or 6. In some of these embodiments, the polyamino moiety may comprises a plurality of —(CH₂)ₓ—NH₂ groups disposed along a polymeric moiety (defined herein as a moiety comprising 2, 3, 4, 5, 6, 7, 8, 9, 10 or more monomer residues). In some embodiments, the polymeric moiety may be selected from a polyamide moiety, such as a peptide moiety, a polyalkylene moiety, or a polysaccharide moiety, among others.

In various embodiments, the polyamino moiety of the polyamino iodinated compounds may correspond to a residue of a carboxyl-substituted polyamino compound (a compound comprising a carboxyl group and a plurality of amino groups). Examples of carboxyl-substituted polyamino compounds include peptides that comprise basic amino acid residues, including residues of amino acids having two or more primary amine groups, such as lysine and ornithine, for example, residues of peptides containing from 2 to 10 lysine and/or ornithine amino acid residues, including polylysine peptides (e.g., dilysine, trilysine, tetralysine, pentalysine, etc.). polyornithine peptides (e.g., diornithine, triornithine, tetraornithine, pentaornithine, etc.), poly(lysine-co-ornithine) peptides, and carboxyl-terminated polyamines such carboxyl-terminated poly(allyl amine), carboxyl-terminated poly(vinyl amine), or carboxyl-terminated chitosan.

Commercially available examples of carboxyl-substituted polyamino compounds also include 16-amino-3-[2-[(4-aminobutyl)(3-aminopropyl)amino]-2-oxoethyl]-12-(3-aminopropyl)-6,9-bis(carboxymethyl)-11-oxo-3,6,9,12-tetraazahexadecanoic acid, L-ornithyl-L-ornithyl-L-ornithine, N²-[1-[N²—[N²—(N-L-valyl-L-alanyl)-L-lysyl]-L-lysyl]-L-prolyl]-L-Lysine, L-Lysyl-L-tryptophyl-L-lysyl-L-lysine, N²,N⁵,N⁵-tris(3-aminopropyl)-L-ornithine, L-lysyl-L-ornithyl-L-lysine, D-lysyl-D-lysyl-D-lysine, glycylglycyl-L-lysylglycylglycyl-L-lysine, N²—[N⁴—[N—[N—(N-glycylglycyl)glycyl]glycyl]-L-lysyl]-L-lysine, L-Lysyl-L-threonyl-L-lysyl-L-lysine, glycylglycyl-L-lysyl-L-lysylglycyl-L-cysteine, L-lysyl-L-arginyl-L-lysyl-L-lysine, L-arginyl-L-lysyl-L-lysyl-L-lysine, L-leucyl-L-lysyl-L-seryl-L-lysyl-L-lysine, L-alanyl-L-methionylglycyl-L-lysyl-L-lysyl-L-lysine, L-lysyl-L-lysyl-L-lysyl-L-arginyl-L-glutamine, L-seryl-L-isoleucyl-L-lysyl-L-lysyl-Llysyl-L-lysine, N²—(N²-L-ornithyl-L-lysyl)-L-lysine, lysyllysyl-lysine, and L-lysyl-L-lysyl-L-lysyl-L-alanine.

As previously noted, in various embodiments, the polyamino iodinated compounds of the present disclosure comprise a polyamino moiety linked to an iodinated moiety through an amide group. The amide group may be the result of a coupling reaction between an amino group of an iodinated amine compound such as one of those described above and a carboxyl group of a carboxyl-substituted polyamino compound, such as one of those described above. The resulting polyamino iodinated compounds therefore comprise a residue of the carboxyl-substituted polyamino compound and a residue of the iodinated amine compound. Stated another way, the polyamino iodinated compounds of the present disclosure may be formed by an amidation reaction in which the carboxyl group of the carboxyl-substituted polyamino compound is reacted with the amino group of the iodinated amine to form an amide bond between the two residues.

In some aspects, the present disclosure pertains to processes of making polyamino iodinated compounds such as those described above.

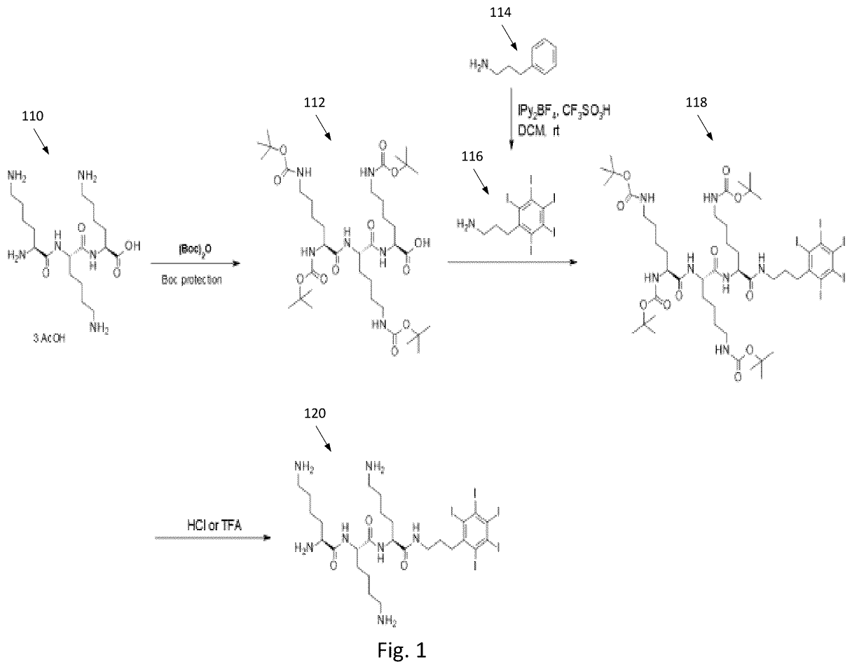

In various embodiments, amino groups of a carboxyl-substituted polyamino compound may be protected with a suitable protective agent. The amino groups are protected for compatibility with other reactants in a subsequent amide coupling reaction described below. For example, amino groups of a carboxyl-substituted polyamino compound may be protected by reaction with di-tert-butyl dicarbonate. In a particular example, and with reference to FIG. 1, four amino groups of trilysine (110), specifically, trilysine acetate (CAS #13184-14-0), are protected using di-tert-butyl dicarbonate (CAS #24424-99-5), thereby forming Boc-protected trilysine (112). This leaves the carboxyl group of the protected compound (t-Boc-protected trilysine) available for amide coupling.

Then, an iodinated amine compound is coupled with the protected carboxyl-substituted polyamino compound in an amide coupling reaction (e.g., via a carbodiimide coupling reagent) to form a protected polyamino iodinated compound. In a particular example, and with reference to FIG. 1, the Boc-protected trilysine (112) is coupled to an iodinated amine compound (3-(2,3,4,5,6-pentaiodophenyl)propan-1-amine) (116), for instance, in the presence of 1-ethyl-3-(3-dimethylaminopropyl)carbodiimide hydrochloride (EDC-HCl) in dimethylformamide (DMF), yielding a pentaiodinated, benzene-based, Boc-protected trilysine compound (118).

As also shown in FIG. 1, 3-phenylpropan-1-amine (CAS #2038-57-5) (114) may be subjected to an iodination with bis(pyridine)iodonium tetrafluoroborate (IPy₂BF₄) and trifluoromethanesulfonic acid(CF₃SO₃H) in dichloromethane at room temperature to obtain the 3-(2,3,4,5,6-pentaiodophenyl)propan-1-amine (116), in which five iodine atoms are attached to the benzene ring. It is noted that less iodine atoms per benzene ring also can be achieved by adjusting the equivalency of IPy₂BF₄ relative to the 3-phenylpropan-1-amine. Moreover, analogous reactions can be performed using 4-phenylbutan-1-amine,

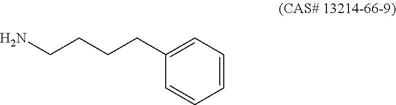 [SMILES: NCCCCc1ccccc1]

and 5-phenylpenta-1-amine,

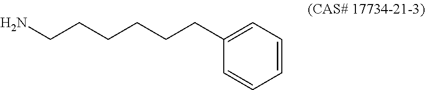 [SMILES: NCCCCCCc1ccccc1]

to form 4-(2,3,4,5,6-pentaiodophenyl)butan-1-amine, and 5-(2,3,4,5,6-pentaiodophenyl)pentan-1-amine, respectively.

Subsequently, deprotection of the protected polyamino iodinated compound is performed (e.g., under acidic conditions), to form a final polyamino iodinated compound, which is useful as a crosslinking compound as discussed below. For example, as shown in FIG. 1, the pentaiodinated, benzene-based, Boc-protected trilysine compound (118) is deprotected under acidic conditions using an acid such as trifluoroacetic acid (TFA) or hydrochloric acid (HCl) to form a pentaiodinated benzene-based trilysine compound (120).

The process described above can be performed using a variety of carboxyl-substituted polyamino compounds and a variety of iodinated amines.

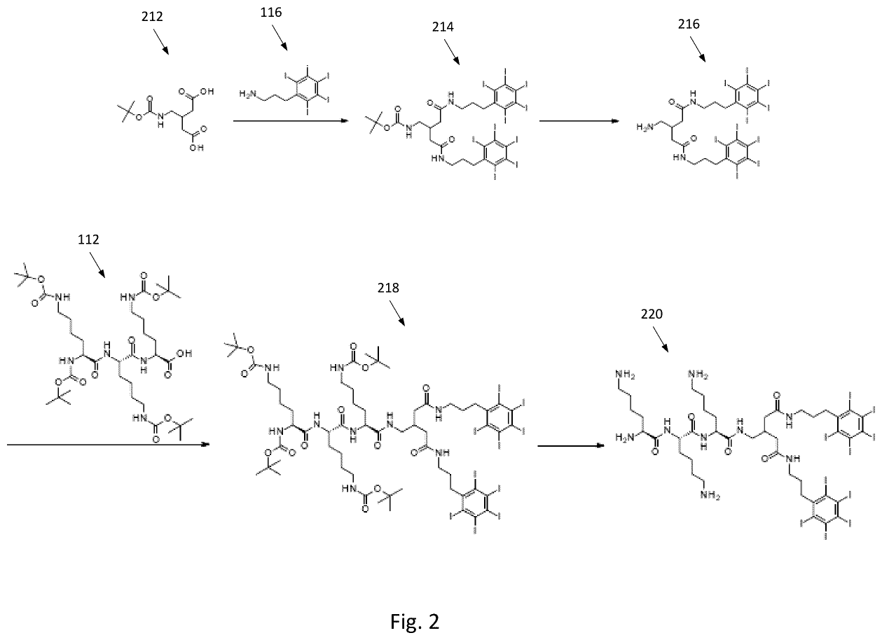

Moreover, the present disclosure provides strategies for increasing the number of iodine atoms that are present in the final polyamino iodinated compound. As an example, and with reference to FIG. 2, commercially available 3-[(tert-butoxycarbonylamino)methyl]pentanedioic acid (CAS #2354255-06-2) (212) may be coupled with an iodinated amine compound, specifically, 3-(2,3,4,5,6-pentaiodophenyl)propan-1-amine (116) to obtain tert-butylN-[4-oxo-2-[2-oxo-2-[3-(2,3,4,5,6-pentaiodophenyl)propylamino]ethyl]-4-[3-(2,3,4,5,6 pentaiodophenyl)propylamino]butyl]carbamate (214), thereby loading two pentaiodinated benzenes into one molecule. Subsequently, Boc deprotection is performed to release the free amine molecule (216), which is further coupled with Boc-protected trilysine (112) to form iodinated Boc-protected trilysine (218), for example, through an EDC coupling reaction. Finally, the iodinated Boc-protected trilysine (218) is deprotected to obtain a final polyamino iodinated compound (220), which contains ten iodine atoms. Note that higher iodine content (more than ten iodine atoms) can also be achieved by using a starting material with more than 2 carboxylic acid groups while maintaining one amino functional group, or by using polyaromatic rings.

As noted above, in several aspects of the present disclosure, a radiopaque crosslinked hydrogel is provided that comprises a crosslinked reaction product of (a) a polyamino iodinated compound such as those described above and (b) a reactive polymer that comprises reactive groups that are reactive with the amino groups of the polyamino iodinated compound.

In various embodiments, the crosslinked products of the present disclosure are visible under fluoroscopy. In various embodiments, such crosslinked products have a radiopacity that is greater than 250 Hounsfield units (HU), beneficially anywhere ranging from 250 HU to 500 HU to 750 HU to 1000 HU or more (in other words, ranging between any two of the preceding numerical values). Such crosslinked products may be formed in vivo (e.g., using a delivery device like that described below), or such crosslinked products may be formed ex vivo and subsequently administered to a subject. Such crosslinked products can be used in a wide variety of biomedical applications, including implants, medical devices, and pharmaceutical compositions.

In various embodiments, the reactive polymer is a reactive multi-arm polymer that comprises a plurality of reactive end groups that are reactive with the amino groups of the polyamino iodinated compound. The reactive end groups of the reactive multi-arm polymer and the amino groups of the polyamino iodinated compound react with one another form a crosslinked product. The reactive multi-arm polymer may be water soluble.

Reactive multi-arm polymers for use herein include those that comprise a plurality of polymer arms (e.g., having two, three, four, five, six, seven, eight, nine, ten, eleven, twelve or more arms), wherein two or more polymer arms of the multi-arm polymers comprise one or more reactive end groups. In some embodiments, compositions containing the reactive multi-arm polymers may be provided in which a percentage of the polymer arms comprising one or more reactive end groups may correspond to between 50% and 100% of the total number of polymer arms in the composition (e.g., ranging anywhere from 50% to 70% to 80% to 90% to 95% to 99% to 100% of the total number of polymer arms). Typical average molecular weights for the reactive multi-arm polymers for use herein are of at least 10 kDa, in some cases ranging from 10 kDa to 50 kDa or more. In various embodiments, the reactive multi-arm polymers for use herein have a melting point of 40° for greater, preferably 450 for greater.

In various embodiments, the polymer arms are hydrophilic polymer arms. Such hydrophilic polymer arms may be composed of any of a variety of synthetic, natural, or hybrid synthetic-natural polymers including, for example, poly(alkylene oxides) such as poly(ethylene oxide) (PEO, also referred to as polyethylene glycol or PEG), poly(propylene oxide) (PPO) or poly(ethylene oxide-co-propylene oxide), poly(N-vinyl pyrrolidone), polyoxazolines including poly(2-alkyl-2-oxazolines) such as poly(2-methyl-2-oxazoline), poly(2-ethyl-2-oxazoline) and poly(2-propyl-2-oxazoline), poly(vinyl alcohol), poly(allyl alcohol), polyhydroxyethyl acrylate, polyhydroxyethyl methacrylate, PEG methyl ether acrylate or PEG methyl ether methacrylate, or PNIPAAM, polysaccharides, and combinations thereof. Such hydrophilic polymer arms may comprise one or more hydrophilic monomers selected from ethylene oxide, N-vinyl pyrrolidone, a 2-alkyl-2-oxazoline monomer, hydroxyethyl acrylate, hydroxyethyl methacrylate, PEG methyl ether acrylate or PEG methyl ether methacrylate, or PNIPAAM.

In some embodiments, the polymer arms extend from a core region. In certain of these embodiments, the core region comprises a residue of a polyol that is used to form the polymer arms. Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, adonitol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

In certain beneficial embodiments, the core region comprises a residue of a polyol that contains two, three, four, five, six, seven, eight, nine, ten or more hydroxy groups. In certain beneficial embodiments, the core region comprises a residue of a polyol that is an oligomer of a sugar alcohol such as glycerol, mannitol, sorbitol, inositol, xylitol, or erythritol, among others.

In certain embodiments, the reactive end groups may be electrophilic groups selected from imidazole esters, imidazole carboxylates, benzotriazole esters, or imide esters, including N-hydroxysuccinimidyl esters. A particularly beneficial reactive end group is an N-hydroxysuccinimidyl ester group. In certain embodiments, the reactive end groups are linked to the polymer arms via hydrolysable ester groups. Hydrolysable ester groups may be selected, for example, from glutarate ester groups, succinate ester groups, carbonate ester groups, or adipate ester groups. In particular embodiments, the polymer arms may be terminated with the following reactive, hydrolysable groups, among others: succinimidyl glutarate groups, succinimidyl succinate groups, succinimidyl carbonate groups, or succinimidyl adipate groups.

Further examples of reactive multi-arm polymers are described, for example, in U.S. Patent Application Nos. 2011/0142936, 2021/0061950, 2021/0061954 and 2021/0061957.

In some aspects of the present disclosure, systems are provided that are configured to deliver (a) a polyamino iodinated compound and (b) a reactive multi-arm polymer that comprises a plurality of reactive end groups that are reactive with the amino groups of the polyamino iodinated compound under conditions such that the polyamino iodinated compound and the reactive multi-arm polymer crosslink with one another. In certain embodiments, those conditions comprise an environment having a basic pH, for example, a pH ranging from about 9 to about 11, typically ranging from about 9.5 to about 10.5, and more typically ranging from about 9.8 to about 10.2. Such systems can be used to form radiopaque crosslinked hydrogels, either in vivo or ex vivo.

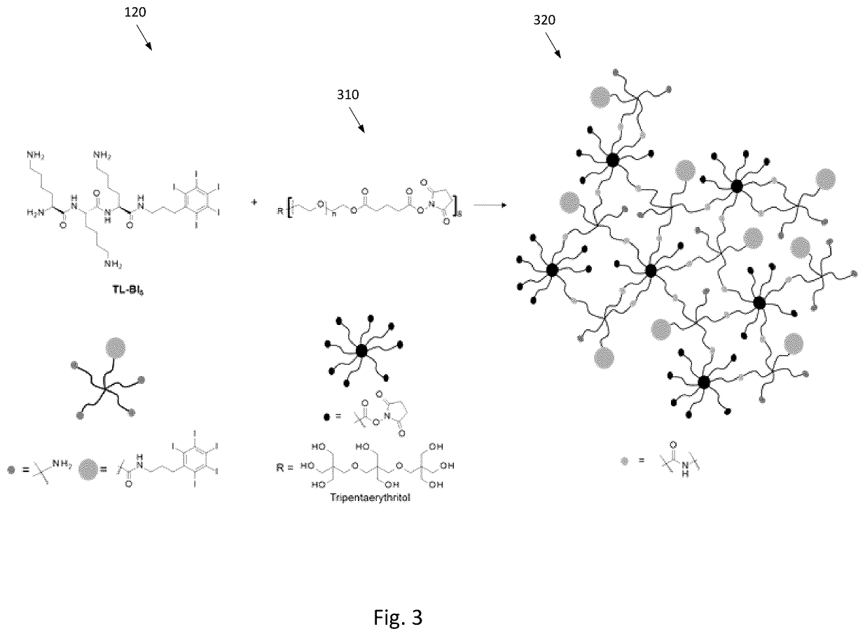

For example, as shown schematically in FIG. 3, a reactive multi-arm polymer like that described above, specifically a reactive multi-arm polymer (310) which comprises a core region R (for example, a tripentaerythritol residue core) and a plurality of hydrophilic polyethylene oxide arms (specifically, eight polyethylene oxide arms, where n ranges, for example, from 25 to 140) having reactive end groups (i.e., succinimidyl glutarate groups) can be crosslinked with a polyamino iodinated compound like that described above, specifically a pentaiodinated benzene-based trilysine compound (120), which comprises amino groups that are reactive with the reactive groups (i.e., succinimidyl glutarate groups) of the reactive multi-arm polymer, by reacting the polyamino iodinated compound with the reactive multi-arm polymer under basic conditions, to form a radiopaque crosslinked product (320), which may be in the form of a hydrogel when hydrated.

An advantage to this approach is that the iodination is separate from the parent polymer, and the multi-arm polymer can be swapped out with N-hydroxysuccinimidyl-ester-functionalized systems having hydrophilic polymer arms other than polyethylene oxide arms, for example, synthetic, natural, or hybrid synthetic-natural hydrophilic polymer arms such as those described above.

In some aspects of the present disclosure, a system is provided that comprises (a) a first composition that comprises a polyamino iodinated compound as described herein and (b) a second composition that comprises a reactive multi-arm polymer that comprises a plurality of reactive end groups that are reactive with the amino groups of the polyamino iodinated compound as described herein.

The first composition may be a first fluid composition comprising the polyamino iodinated compound or a first dry composition that comprises the polyamino iodinated compound, to which a suitable fluid such as water for injection, saline, etc. can be added to form a first fluid composition. In addition to the polyamino iodinated compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents, including therapeutic agents, imaging agents, colorants, tonicity adjusting agents, suspension agents, wetting agents, and pH adjusting agents as described below.

In some embodiments, the polyamino iodinated compound (120) is initially combined with the reactive multi-arm polymer (310) at an acidic pH at which crosslinking between the reactive groups of the reactive multi-arm polymer (310) and the amino groups of the polyamino iodinated compound (120) is suppressed. Then, when crosslinking is desired, a pH of the mixture of the polyamino iodinated compound (120) and the reactive multi-arm polymer (310) is changed from an acidic pH to a basic pH, leading to crosslinking between same, thereby forming the crosslinked product (320).

In particular embodiments, the system comprises (a) a first composition that comprises a polyamino iodinated compound as described hereinabove, (b) a second composition that comprises a reactive multi-arm polymer as described hereinabove, and (c) a third composition, specifically, an accelerant composition, that contains an accelerant that is configured to accelerate crosslinking reaction between the polyamino iodinated compound and the reactive multi-arm polymer.

The first composition may be a first fluid composition comprising the polyamino iodinated compound that is buffered to an acidic pH or a first dry composition that comprises the polyamino iodinated compound and acidic buffering composition, to which a suitable fluid such as water for injection, saline, etc. can be added to form a first fluid composition comprising the polyamino iodinated compound that is buffered to an acidic pH. In some embodiments, for example, the acidic buffering composition may comprise monobasic sodium phosphate, among other possibilities. The first fluid composition comprising the polyamino iodinated compound may have a pH ranging, for example, from about 3 to about 5, typically ranging from about 3.5 to about 4.5, and more typically ranging from about 3.8 to about 4.2. In addition to the polyamino iodinated compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer from which a fluid composition is formed, for example, by the addition of a suitable fluid such as water for injection, saline, or the first fluid composition comprising the polyamino iodinated compound that is buffered to an acidic pH. In addition to the reactive multi-arm polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyamino iodinated compound that is buffered to an acidic pH and the second composition comprises a dry composition that comprises the reactive multi-arm polymer. The first composition may then be mixed with the second composition to provide a prepared fluid composition that is buffered to an acidic pH and comprises the polyamino iodinated compound and the reactive multi-arm polymer. In a particular example, a syringe may be provided that contains a first fluid composition comprising the polyamino iodinated compound that is buffered to an acidic pH, and a vial may be provided that comprises a dry composition (e.g., a powder) that comprises the reactive multi-arm polymer. The syringe may then be used to inject the first fluid composition into the vial containing the reactive multi-arm polymer to form a prepared fluid composition that contains the polyamino iodinated compound and the reactive multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11, typically ranging from about 9.5 to about 10.5, and more typically ranging from about 9.8 to about 10.2. In addition to the above, the fluid accelerant composition may further comprise additional agents, including those described below.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 1111n, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present in the polyamino iodinated compound) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol©).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

A prepared fluid composition that is buffered to an acidic pH and comprises the polyamino iodinated compound and the reactive multi-arm polymer as described above, and a fluid accelerant composition that is buffered to basic pH as described above, may be combined form radiopaque crosslinked hydrogels, either in vivo or ex vivo.

In various embodiments, a system is provided that include one or more delivery devices for delivering first and second compositions to a subject.

In some embodiments, the system may include a delivery device that comprises a first reservoir that contains a first composition that comprises a polyamino iodinated compound as described above and a second reservoir that contains a second composition that comprises a reactive multi-arm polymer that comprises a plurality of reactive end groups that are reactive with the amino groups of the polyamino iodinated compound as described above.

In some embodiments, the system may include a delivery device that comprises a first reservoir that contains a first composition that comprises the polyamino iodinated compound and the reactive multi-arm polymer and is buffered to an acidic pH, such as the prepared fluid composition previously described, and a second reservoir that contains second composition, such as the fluid accelerant composition previously described. In either case, during operation, the first composition and second composition are dispensed from the first and second reservoirs and combined, whereupon the polyamino iodinated compound and the reactive multi-arm polymer and crosslink with one another to form a radiopaque crosslinked hydrogel.

Regardless of the first and second compositions selected, in particular embodiments, the system may include a delivery device that comprises a double-barrel syringe, which includes first barrel having a first barrel outlet, which first barrel contains the first composition, a first plunger that is movable in the first barrel, a second barrel having a second barrel outlet, which second barrel contains the second composition, and a second plunger that is movable in the second barrel.

In some embodiments, the device may further comprise a mixing section having a first mixing section inlet in fluid communication with the first barrel outlet, a second mixing section inlet in fluid communication with the second barrel outlet, and a mixing section outlet. In some embodiments, the device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions interact and ultimately crosslink to form a radiopaque crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a radiopaque crosslinked hydrogel is ultimately formed at the administration location.

After administration, the compositions of the present disclosure can be imaged using a suitable imaging technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

As noted above, in several aspects of the present disclosure, a radiopaque crosslinked hydrogel is provided that comprises a crosslinked reaction product of (a) a polyamino iodinated compound such as those described above and (b) a reactive polymer that comprises reactive groups that are reactive with the amino groups of the polyamino iodinated compound.

In various embodiments, the reactive polymer is a polysaccharide that comprises a plurality of reactive groups that are reactive with the amino groups of the polyamino iodinated compound, wherein a crosslinked product is formed between the reactive groups of the polysaccharide and the amino groups.

In some of these embodiments, a radiopaque crosslinked polysaccharide is provided in which the polysaccharide and the polyamino iodinated compound are covalently linked through amide groups. For example, the radiopaque crosslinked polysaccharide may be formed, for example, by an amide-forming reaction between the amino groups of the polyamino iodinated compound and carboxylic acid groups of a carboxylic-acid-containing polysaccharide. Carboxylic-acid-containing polysaccharides include those that contain one or more uronic acid species, such as galacturonic acid, glucuronic acid and/or iduronic acid. Particular examples of carboxylic-acid-containing polysaccharides include alginic acid, hyaluronic acid, pectin, agaropectin, carrageenan, gellan gum, gum arabic, guar gum, xanthan gum, and carboxymethyl cellulose moieties. In embodiments where the carboxylic-acid-containing polysaccharide is hyaluronic acid, the carboxylic-acid-containing polysaccharide may be non-animal stabilized hyaluronic acid. In some embodiments, the carboxylic-acid-containing polysaccharides may have a number average molecular weight ranging from 1 kDa to 8000 kDa, for example ranging anywhere from 1 kDa to 2.5 kDa to 5 kDa to 10 kDa to 25 kDa to 50 kDa to 100 kDa to 250 kDa to 500 kDa to 1000 kDa to 2000 kDa to 8000 kDa (in other words, ranging between any two of the preceding numerical values).

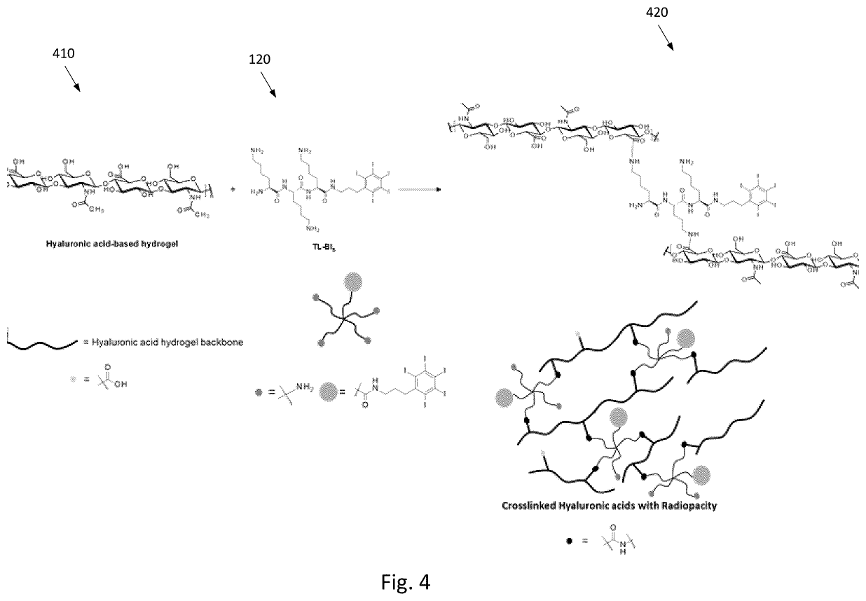

Radiopaque crosslinked hydrogels in accordance with the present disclosure may be formed by an amide coupling reaction between a carboxylic-acid containing polysaccharide, such as one of those described above, among others, and a polyamino iodinated compound, such as one of those described above, among others. Such a coupling reaction may be performed using a suitable coupling reagent, for instance, a carbodiimide coupling reagent. In a particular example shown in FIG. 4, hyaluronic acid (410) is employed as the carboxylic-acid containing polysaccharide and the pentaiodinated benzene-based trilysine compound (120) of FIG. 1 is the polyamino iodinated compound. 1-ethyl-3-(3-dimethylaminopropyl)carbodiimide (EDC) may be used as a coupling reagent. The resulting radiopaque crosslinked polysaccharide product (420) will be in the form of a hydrogel when hydrated.

In various embodiments, such radiopaque crosslinked hydrogels have a radiopacity that is greater than 250 Hounsfield units (HU), beneficially anywhere ranging from 250 HU to 500 HU to 750 HU to 1000 HU or more in various embodiments.

Such radiopaque crosslinked polysaccharides may be in any desired form, including a slab, a cylinder, a coating, or a particle. In some embodiments, the radiopaque crosslinked polysaccharide is dried and then granulated into particles of suitable size. Granulating may be by any suitable process, for instance by grinding (including cryogrinding), crushing, milling, pounding, or the like. Sieving or other known techniques can be used to classify and fractionate the particles. Radiopaque crosslinked polysaccharide particles formed using the above and other techniques may varying widely in size, for example, having an average size ranging from 50 to 950 microns.

In addition to a radiopaque crosslinked polysaccharide as described above, radiopaque crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the radiopaque crosslinked polysaccharide hydrogel to a subject. Such systems may include one or more of the following: a syringe barrel, which may or may not contain a radiopaque crosslinked polysaccharide as described herein; a vial, which may or may not contain a radiopaque crosslinked polysaccharide as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the radiopaque crosslinked polysaccharide may be provided in dry form (e.g., powder form) or in a form that is ready for injection, such as an injectable hydrogel form.

The radiopaque crosslinked polysaccharide hydrogel compositions described herein can be used for a number of purposes.

For example, radiopaque crosslinked polysaccharide hydrogel compositions can be injected to provide spacing between tissues, radiopaque crosslinked polysaccharide hydrogel compositions can be injected (e.g., in the form of blebs) to provide fiducial markers, radiopaque crosslinked polysaccharide hydrogel compositions can be injected for tissue augmentation or regeneration, radiopaque crosslinked polysaccharide hydrogel compositions can be injected as a filler or replacement for soft tissue, radiopaque crosslinked polysaccharide hydrogel compositions can be injected to provide mechanical support for compromised tissue, radiopaque crosslinked polysaccharide hydrogel compositions be injected as a scaffold, and/or radiopaque crosslinked polysaccharide hydrogel compositions can be injected as a carrier of therapeutic agents in the treatment of diseases and cancers and the repair and regeneration of tissue, among other uses.

After administration, the radiopaque crosslinked polysaccharide hydrogel compositions of the present disclosure can be imaged using a suitable imaging technique.

As seen from the above, the radiopaque crosslinked polysaccharide hydrogel compositions of the present disclosure may be used in a variety of medical procedures, including the following, among others: a procedure to implant a fiducial marker comprising a radiopaque crosslinked polysaccharide hydrogel, a procedure to implant a tissue regeneration scaffold comprising a radiopaque crosslinked polysaccharide hydrogel, a procedure to implant a tissue support comprising a radiopaque crosslinked polysaccharide hydrogel, a procedure to implant a tissue bulking agent comprising a radiopaque crosslinked polysaccharide hydrogel, a procedure to implant a therapeutic-agent-containing depot comprising a radiopaque crosslinked polysaccharide hydrogel, a tissue augmentation procedure comprising implanting a radiopaque crosslinked polysaccharide hydrogel, a procedure to introduce a radiopaque crosslinked polysaccharide hydrogel between a first tissue and a second tissue to space the first tissue from the second tissue.

The radiopaque crosslinked polysaccharide hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Radiopaque crosslinked polysaccharide compositions in accordance with the present disclosure also include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A radiopaque crosslinked hydrogel composition comprising a crosslinked reaction product of (a) a polyamino iodinated compound and (b) a reactive polymer that comprises a plurality of reactive groups that are reactive with the amino groups of the polyamino iodinated compound.

2. The radiopaque crosslinked hydrogel composition of claim 1, wherein the polyamino iodinated compound comprises a polyamino moiety that is linked to an iodinated moiety through an amide group.

3. The radiopaque crosslinked hydrogel composition of claim 2, wherein the iodinated moiety is an iodinated aromatic moiety.

4. The radiopaque crosslinked hydrogel composition of claim 3, wherein the iodinated aromatic moiety comprises a monocyclic or multicyclic aromatic structure that is substituted with one or more iodine atoms.

5. The radiopaque crosslinked hydrogel composition of claim 4, wherein the monocyclic or multicyclic aromatic structure is further substituted with one or more hydroxy-containing groups independently selected from hydroxy groups and C₁-C₄-hydroxyalkyl groups.

6. The radiopaque crosslinked hydrogel composition of claim 2, wherein polyamino moiety comprises a plurality of —(CH₂)ₓ—NH₂ groups where x is 0, 1, 2 3, 4, 5 or 6.

7. The radiopaque crosslinked hydrogel composition of claim 1, wherein the polyamino iodinated compound comprises a residue of a peptide oligomer that comprises from 2 to 10 lysine and/or ornithine amino acid residues and a residue of an iodinated amine compound.

8. The radiopaque crosslinked hydrogel composition of claim 7, wherein the iodinated amine compound comprises a monocyclic or multicyclic aromatic structure substituted with (a) an amine substituent, (b) one or more iodine atoms and (c) optionally, one or more hydroxy-containing groups independently selected from hydroxy groups and C₁-C₄-hydroxyalkyl groups.

9. The radiopaque crosslinked hydrogel composition of claim 8, wherein the amine substituent is selected from an amino (—NH₂) group and an amino-C₁-C₆-alkyl group.

10. The radiopaque crosslinked hydrogel composition of claim 1, wherein the reactive polymer is a reactive multi-arm polymer that comprises a plurality of hydrophilic polymer arms having reactive end groups that are reactive with amino groups of the polyamino iodinated compound.

11. The radiopaque crosslinked hydrogel composition of claim 10, wherein the hydrophilic polymer arms comprise one or more hydrophilic monomers selected from ethylene oxide, N-vinyl pyrrolidone, oxazoline monomers, hydroxyethyl acrylate, hydroxyethyl methacrylate, PEG methyl ether acrylate or PEG methyl ether methacrylate, or PNIPAAM.

12. The radiopaque crosslinked hydrogel composition of claim 10, wherein the reactive end groups are linked to the hydrophilic polymer arms by a hydrolysable ester and/or wherein the reactive end groups are electrophilic groups.

13. The radiopaque crosslinked hydrogel composition of claim 1, wherein the reactive polymer comprises carboxylic acid groups that react with the amino groups of the polyamino iodinated compound to form amide linkages.

14. The radiopaque crosslinked hydrogel composition of claim 13, wherein the reactive polymer is a carboxylic-acid-containing polysaccharide.

15. The radiopaque crosslinked hydrogel composition of claim 4, wherein the carboxylic-acid-containing polysaccharide is hyaluronic acid.

16. The radiopaque crosslinked hydrogel composition of claim 1, comprising radiopaque particles that comprise the crosslinked reaction product.

17. The radiopaque crosslinked hydrogel composition of claim 1, further comprising one or more additional agents selected from therapeutic agents, imaging agents, colorants, tonicity adjusting agents, suspension agents, wetting agents, and pH adjusting agents.

18. A system for forming a hydrogel composition that comprises (a) a polyamino iodinated compound and (b) a reactive polymer that comprises a plurality of reactive groups that are reactive with the amino groups of the polyamino iodinated compound.

19. The system of claim 18, wherein the system comprises a first composition that comprises the polyamino iodinated compound, a second composition that comprises the reactive polymer, and an optional accelerant composition.

20. A method of treatment comprising administering to a subject a mixture that comprises a polyamino iodinated compound and a reactive multi-arm polymer that comprises a plurality of hydrophilic polymer arms having reactive end groups that are reactive with amino groups of the polyamino iodinated compound under conditions such that the polyamino iodinated compound and the reactive multi-arm polymer crosslink after administration.
